(19) United States
(12) Patent Application Publication
(10) Pub. No.: US 2010/0049074 A1
(43) Pub. Date: Feb. 25, 2010

(54) CATEGORIZING SONGS ON A PHYSIOLOGICAL EFFECT

(75) Inventor: Hubertus Maria Rene Cortenraad, Maastricht (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/815,132

(22) PCT Filed: Jan. 24, 2006
(86) PCT No.: PCT/IB2006/050257

§ 371 Date: Jul. 31, 2007

(30) Foreign Application Priority Data
Jan. 31, 2005 (EP) 05100610.4

(51) Int. Cl. A61B 5/0484; G06F 17/00; G06F 3/048

(52) U.S. Cl. 600/544; 700/90; 715/764

(57) ABSTRACT

A system (100) for categorizing songs on a physiological effect of the song on a user includes a storage medium (120) for storing songs, a memory (140) for storing for a predetermined physiological state of a user an associated predetermined first selection criterion, a rendering system (150) for rendering songs, and a sensor (130) for determining a biological parameter of the user representative of a physiological state of the user. A processor (110) tests a selected song for a physiological effect. The selected song is rendered a plurality of times. Each time, the sensor obtains at least one measurement, and the first criterion is used to determine a respective indicator indicating whether rendering of the song has a positive effect. Next, a second predetermined criterion is used to, based on a plurality of the determined indicators, determine a suitability of the song for bringing the user closer to the predetermined physiological state.

### FIELD OF THE INVENTION

The invention relates to a system for and a method of categorizing songs on a physiological effect of the song on a user.

The invention further relates to a computer program product implementing such a method.

### BACKGROUND OF THE INVENTION

US 2003/0060728 describes that music can control how a person acts or feels. A person's mood or state of mind can frequently be inferred from readily measurable physiological conditions, such as pulse, blood pressure, temperature and brain wave activity. Sensors are used to detect the physiological condition. The system operates in two modes: a training mode and a playback mode. In the training mode, the user listens to a piece of music. The physiological condition of the user is measured. At the end of playing of the piece, the user is requested to specify the physiological state he is in. Both the measured state and the user-specified state are stored. This is in principle repeated for each piece of music. In the playback mode, music is selected according to information entered by the user or the measured physiological state. Examples of categories a user may specify are: sleepy, energized or peaceful.

A disadvantage of the known system is that each song needs to be trained. Although this in theory could provide an accurate system, particularly for critical physiological states, such as a transition from awake to asleep, the perception of the user of the effect of the song may not be accurate. Moreover, the training is time consuming.

### SUMMARY OF THE INVENTION

It is an object of the invention to provide an improved system and method of the kind set forth suitable for automatic operation.

To meet the object of the invention, the system for categorizing songs on a physiological effect of the song on a user includes:

a storage medium for storing a plurality of songs;

a memory for storing for at least one predetermined physiological state of a user an associated predetermined first selection criterion;

a rendering system for rendering songs retrieved from the storage;

a sensor for determining a biological parameter of the user representative of a physiological state of the user;

a processor for, under control of a program, testing a selected song for a physiological effect on the user, by:
a plurality of times: causing the selected song to be rendered; and for each rendering of the song: using the sensor to obtain a measurement at least once during the rendering of the song, and using the first predetermined criterion to determine, in dependence on the measurement, a respective indicator indicating whether rendering of the song has a positive effect on bringing the user closer to the predetermined physiological state; and
using a second predetermined criterion to, based on a plurality of the determined indicators for the song, determine a suitability of the song for bringing the user closer to the predetermined physiological state.

In the system according to the invention, two criteria are used. The first criterion is used to determine whether rendering of a song has a positive effect. The criterion is specific for the physiological state. The system may support more than one physiological state, each having a respective first criterion. Applying the criterion results in an indicator indicating whether playing of the song has a positive effect on the physiological state. Using physiological state-specific criteria makes the system suitable for automatic detection. The inventor has realized that once playing a song may not give a reliable outcome on the effect. For example, if a song is being rendered that in principle would aid in bringing the user into sleep, but actually during the rendering somebody slams a door in the vicinity, that may not be the case in that instance. This is overcome by using a second criterion that based on the effect determined for a plurality of times of rendering the song comes to a final decision on the effect of the song. In this way, spurious effects can be automatically eliminated. It will be appreciated that with song is meant any piece of music/audio reproducible by an audio system. This typically covers songs with lyrics as well as music. It also covers artificial sounds.

According to a measure as defined in the dependent claim 2, the effect of rendering a song once is based on at least two time-sequential measurements, where the measured change in the physiological state is the decisive criterion. For example, if a person at the start of the song is awake but at the end is half asleep this is used as an indicator that the song is suitable for the physiological state ‘asleep’, whereas actually both the start and the end condition differ from the ‘asleep’ state. In an embodiment, the state is measured at the start and at the end of the song. Comparing these two measurements will in most situations provide a reliable indication of the effect of the song. Preferably, the state is measured frequently during the rendering. In this way also undesirable peaks in the state can be detected, for example a song may overall have a positive effect but also has a negative effect e.g. a short period with loud music in an overall relaxing song. Such a peak may be an indicator that the song is less suitable.

According to a measure as defined in the dependent claim 3, the sensor is a brain-wave sensor for determining a frequency of brainwaves of the user. Such a sensor is highly suitable for determining whether or not a person is asleep or alert. It can thus be useful for selecting relaxing music (e.g. to help a user to relax or fall asleep) or music intended to keep a user alert (e.g. when driving car) or awakening a user (e.g. after the alarm has gone off).

According to a measure as defined in the dependent claim 4, the sensor is of a contact-free type. Using such a sensor is unobtrusive. The sensor may be embedded in the sleeping cushion or head-rest of a seat, such as a car seat.

According to a measure as defined in the dependent claim 5, the predetermined physiological state is sleep and the first criterion includes whether a brainwave frequency has decreased during rendering of the song. A decrease of frequency is a good indicator of the user relaxing.

According to a measure as defined in the dependent claim 6, the predetermined physiological state is alert and the first criterion includes whether a brainwave frequency has increased during rendering of the song. An increase of frequency is a good indicator of the user becoming more alert.

According to a measure as defined in the dependent claim 7, the first criterion is dependent on an actual physiological state at that moment, possibly in addition to the change of the state measured during the rendering. An example of this is given in the dependent claim 8, where, if the desired physiological state has already been reached (as indicated by the actual physiological state, the fact that no substantial change is detected in the measurements during the rendering of the song is regarded as a positive indicator.

According to a measure as defined in the dependent claim 9, the second criterion includes that a predetermined successive number of times during the rendering a same song the first indicator has indicated that rendering of the song has a positive effect on bringing the user closer to the predetermined physiological state.

According to a measure as defined in the dependent claim 10, the memory is arranged to store for the predetermined physiological state an associated playlist of songs for bringing the user closer to the predetermined physiological state.

According to a measure as defined in the dependent claim 11, the processor is programmed to, based on the determined suitability, determining whether the song should be added to and/or maintained on the playlist. In this way automatically a playlist can be created. Subsequent rendering of songs on the playlist aids in reaching the desired physiological state.

According to a measure as defined in the dependent claim 12, the memory includes for songs on the playlist an associated suitability indicator and the processor is programmed to determine a relative rate of rendering of a song on the playlist in dependence on the associated suitability factor. In this way, the playlist is more varied and can include more songs making it more attractive for a user.

According to a measure as defined in the dependent claim 13, the memory includes a candidate playlist of songs being tested for acceptance on the playlist; the candidate play-list including for songs on the candidate playlist associated information including at least the indicator indicating whether rendering of the song has a positive effect on relaxing the user. A new song may first be entered on a candidate list. It will be appreciated that the list of candidate songs may be incorporated in the playlist itself, where the candidates songs are marked as not yet (fully) accepted. In this way, the song may be played automatically without any further active selection or involvement of the user.

According to a measure as defined in the dependent claim 14, the processor is programmed to use the brain-wave sensor to determine at least one of the following physiological states:

the user is falling asleep,

the user is asleep,

the user is awakening,

the user is alert;

and, in response to determining so, stop rendering of songs or start rendering of songs.

According to a measure as defined in the dependent claim 15, the system includes a user interface for enabling a user to select the song for testing. The user may directly select individual songs, may select the songs by specifying an existing playlist, or by providing selection criteria (e.g. genre, artist, etc.).

According to a measure as defined in the dependent claim 16, the rendering system includes speakers for generating spatially localized sound, such as an earphone, headphone or sound-beamer. In this way, the effect of the system can be limited to a single user. This is particularly advantageous if the song may not have the same effect for each user.

These and other aspects of the invention are apparent from and will be elucidated with reference to the embodiments described hereinafter.

### BRIEF DESCRIPTION OF THE DRAWINGS

In the drawings:

FIG. 1 shows a block diagram of an embodiment according to the invention,

FIG. 2 shows a flow-chart of a method according to the invention;

FIG. 3 shows a flow-chart of a further embodiment according to the invention; and

FIG. 4 shows an exemplary field stored in a play-list.

### DETAILED DESCRIPTION OF THE PREFERRED EMBODIMENT

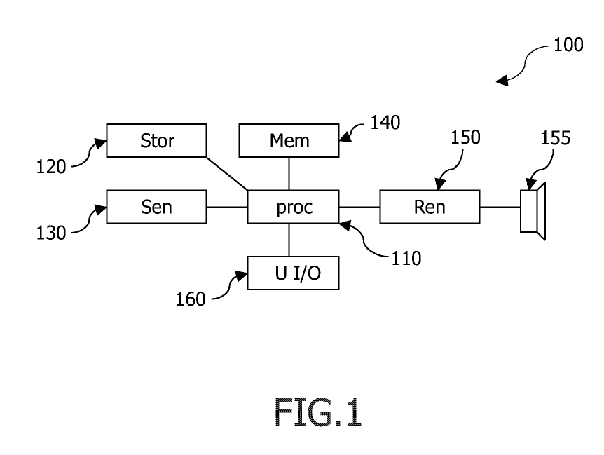

FIG. 1 shows a block diagram of an embodiment of the system 100 according to the invention. The system is used for categorizing songs on a physiological effect of the song on a user. The purpose is to select in this way songs that can later on be used to influence a physiological state of the user. The system operates automatically. It includes at least one sensor 130 for determining a biological parameter of the user representative of a physiological state of the user. In principle any physiological state of the user that can be influenced by music (and whose effect can be measured) may be influenced in the system. Examples of such states are states of mind, like sleeping, drowsiness, relaxed, alert/focused, etc. as well as states (moods) like happiness/sadness, etc. Any suitable sensor may be used. For example, the sensor 130 may measure a heart-rate, blood-pressure, sweat level on the skin, etc. Preferably also the brain-wave frequency may be measured. Particularly, the brain-wave frequency is a good measure for determining whether as user is asleep, relaxed, or alert. If more than one biological parameter is measured, each of those biological parameters may be measured with physically separate sensors; alternatively a measuring device may be used that integrates a plurality of those sensors. Preferably, the sensors are as unobtrusive as possible. For example, sensors may be used that do not need to be attached to the human body, that are of a contact-free type and thus operate remote from the body. Significant progress has already been made with developing such types of sensors. For example, for military applications contact-less brainwave sensors have already been developed. Also sensors exist in cars that can detect that a user is falling asleep. Any such sensor may be used.

Clearly, the system can play (=render) songs. In the embodiment, the songs are stored in a storage medium 120. This may be any suitable storage medium, like optical storage (e.g. a CD, DVD, Blu-Ray disc), magnetic storage (e.g. hard disc), solid state (e.g. flash memory). This storage may be of a removable type or fixed built-in. In the latter case it is preferred that new songs can be loaded into the system, e.g. via an I/O interface (e.g. USB, or memory card interface, wired or wireless network connection, etc.). In FIG. 1 such an interface is not shown. The system also includes a rendering system 150 for rendering songs retrieved from the storage. The rendering system 150 also includes means for generating the music. Shown are loudspeakers 155. Any suitable type of loudspeakers may be used. These include conventional loudspeakers of stereo systems or surround-sound systems, headphones, ear-phones, a loudspeaker that can be placed under a cushion while resting on a bed, beam-forming speaker systems, etc. It will be appreciated that some of these speakers are highly suitable for providing sound to a single user, so that that user can hear songs targeted towards a personally desired physiological state, whereas other people hear nothing or are provided with their own selection of songs, possibly targeted towards another physiological state.

The system includes a memory 140 for storing for at least one predetermined physiological state of a user an associated predetermined first selection criterion. If the system supports more than one physiological state it stores for each of those states a respective first selection criterion. The memory 140 may be of any suitable type, preferably including at least partly a non-volatile memory for storing parameters also during periods of no power. Suitable memory includes rewriteable optical disc, hard disk, flash memory, etc. A processor 110 is used to test a selected song for a physiological effect on the user. Any suitable type of processor may be used, such as a conventional microprocessor used in PCS, or digital signal processors (DSPs) frequently used in consumer electronics audio/video equipment. The processor 110 is operated under control of a program. The program is typically loaded from a storage (e.g. storage 120 or memory 140).

The program executes two main steps:
1. It causes the selected song to be rendered by the rendering system 150 and collects at least one measurement from the sensor 130. It then uses the first predetermined criterion stored in memory 140 to determine an indicator indicating whether rendering of the song has a positive effect on bringing the user closer to the predetermined physiological state. Next, it stores the indicator in association with the selected song (e.g. in memory 140 or in storage 120). The indicator is representative of the effect of this specific rendering session. The processor performs this rendering and using of the first criterion at least twice; each time giving a respective first indicator. It will be appreciated that storing of the first criterion in memory 140 also covers the situation wherein the criterion is fixed coded into the program (e.g. the program is loaded from the memory 140).
2. It uses a second predetermined criterion to, based on a plurality of the determined indicators for the song, determine a suitability of the song for bringing the user closer to the predetermined physiological state. The second criterion may also be stored in memory 140 but may also be fixed encoded in the program.

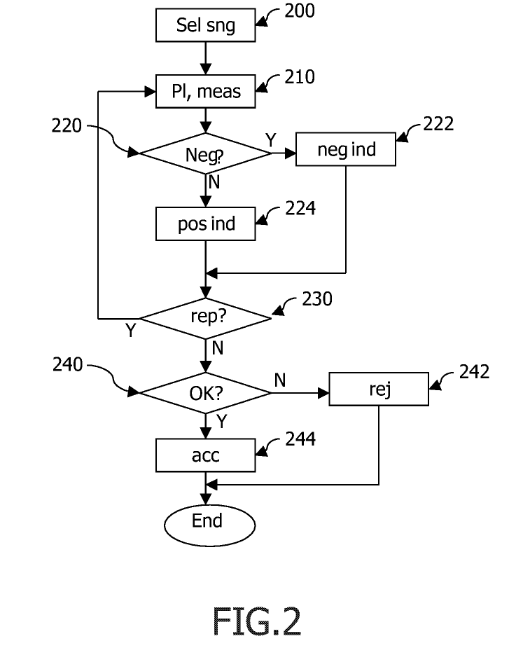

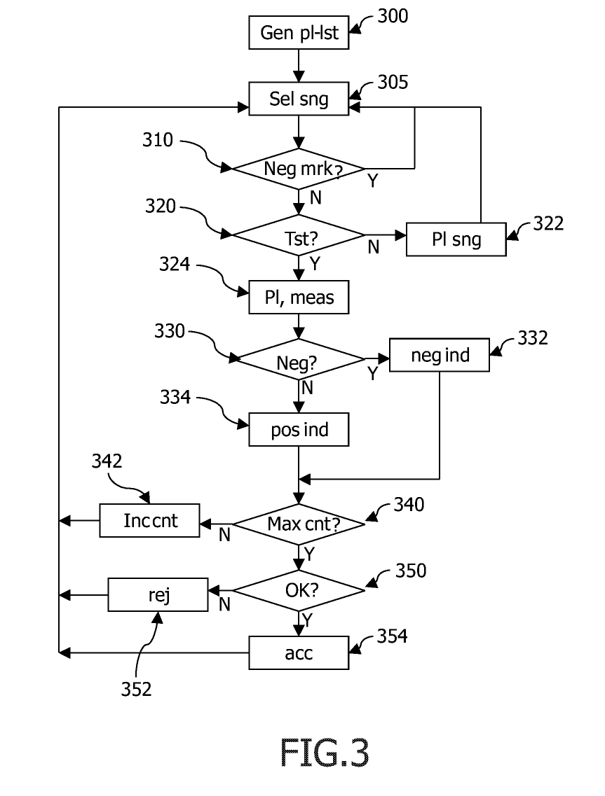

FIG. 2 shows an embodiment of the method according to the invention. In step 200 a songs is selected for testing. The selection may be performed in any suitable way. For example, a user interface 160 of the system may be used to enable a user to select a song for testing. Any suitable user interface may be used, such a using a display to present options to the user and a remote control, mouse, keyboard, remote control or buttons on the device to enable the user to specify its choice. Also voice control may be used. The selection may also implicit to another action of the user, for example the user inserts a CD and all songs are tested by automatically selecting them in turn. The more detailed embodiment of FIG. 3 shows that in step 300 a play-list of songs to be tested is generated. Such a play-list may be generated in any suitable way, for example by the user selecting songs from a library of songs or by the user specifying selection criteria, such as music genre, artist, etc. In situations where the user has selected more than one song for testing, the system preferably automatically selects a single song to be tested at a time. This selection may take any suitable form, e.g. sequential selection, shuffling, etc. Preferably, the user can select such a selection mode. In step 210 of FIG. 2 and step 324 of FIG. 3 the selected song is directed to the rendering system 150 for rendering. In steps 210 and 324, also the sensor 130 is used to collect at least one measurement during the rendering. In step 220 and 330, respectively, the first predetermined criterion stored in memory 140 is used to determine an indicator indicating whether rendering of the song has a positive effect on bringing the user closer to the predetermined physiological state. It then stores the indicator in association with the selected song (e.g. in memory 140 or in storage 120). Box 222 and 332, respectively, show that a negative indication is stored. Box 224 and 334, respectively, show that a positive indicator is stored.

The following steps illustrate the second criterion. In step 230 it is checked whether the song has been tested enough. According to the invention, each song is tested at least twice. It will be appreciated that if a song is tested always exactly twice, that then step 230 can be implemented in a simple way, without an explicit test. FIG. 3 shows an embodiment suitable for testing more than twice. In this embodiment a counter is used. The counter is incremented in box 342. In box 340 it is checked whether the counter has reached a predetermined maximum (e.g. four times testing). It will be appreciated that initially the counter for a song to be tested needs to be set to an initial value, e.g. zero. In step 240 of FIG. 2 and step 350 of FIG. 3, a decision is taken whether or not the song is suitable. Box 242 and box 352, respectively, show that a negative outcome (unsuitable) is stored. Box 244 and 354, respectively, shows that a positive outcome (suitable) is stored.

In a preferred embodiment, the processor is programmed to use the sensor at least twice during the rendering of the song to obtain at least two time-sequential measurements of the biological parameter. It will be appreciated that also many more measurements may be taken, e.g. at fixed intervals of a couple of seconds or substantially continuous. The second criterion is then based on whether or not the rendering of the song has a positive effect on bringing the user closer to the predetermined physiological state in dependence on a change of the measurements. For example, if two measurements are taken (e.g. one at the beginning of the song and one at the end of the song) the criterion can be that an improvement has occurred.

Taking as an example the brainwave frequency of a user as biological measurement, then it is known that the following relationship exists between the brainwave frequency and the state of mind of the user:

| State name | Frequency range | State of mind |
|---|---|---|
| Delta | 0.5 Hz.-4 Hz. | deep sleep |
| Theta | 4 Hz.-8 Hz. | Drowsiness (also first stage of sleep) |
| Alpha | 8 Hz.-14 Hz. | Relaxed but alert |
| Beta | 14 Hz.-30 Hz. | Highly alert and focused |

If the user now wants songs selected that are suitable for that person to fall asleep (desired physiological state is ‘asleep’), a suitable first indicator is that the brainwave frequency has lowered during the playing of the song. If the desired physiological state is ‘highly alert’ (e.g. the user wants to select songs to be played while driving a car), a suitable first indicator is that the brainwave frequency has increased during the playing of the song. The latter physiological state also illustrates that an alternative or additional first criterion is that the desired physiological state has already been achieved and that no negative change (e.g. no substantial change at all or even a small improvement) has been measured during the rendering session of the song. A person skilled in the art can easily decide for which physiological states a stable situation is a good indicator. It will be appreciated that for most persons in deep sleep a stable measurement is not providing any useful information.

A suitable second criterion for above examples is that the first criterion has been met for three successive renderings of the same song. It will be appreciated that a positive effect can also be that no negative effect has been detected (e.g. frequency has not changed substantially).

The reliability of the system can be improved by frequently monitoring the effect during the rendering. In this way also short negative effects can be detected. For example, somewhere in the middle of a song a relatively loud sound occurs, which could slightly wake-up the user and thus would make the song less suitable for falling asleep, even if the entire song might still have a positive effect. So, the outcome of the first criterion may be a suitable or not suitable ranking, but may also offers a more varied assessment like moderately suitable (e.g. overall positive but with a negative intermediate effect). All these aspects may be take into account for the second criterion. The outcome of the second criterion may thus also be “black or white” (i.e. not suitable or suitable) but may also be more varied.

It is desired that the system determines the real correlation between rendering of the song and the effect on the physiological state and tries to eliminate disturbances that may influence that. For example, if songs are selected for falling asleep (i.e. the person lies already in bed and listens to the songs), then a negative effect may first be measured if the user is awakened by something else than the music. This could be any external trigger, such as a loud sound generated in the street, or a push from the partner. Some of such triggers (e.g. loud sounds) the system may be able to detect (e.g. by using a microphone and comparing the received sound to the rendered sound). If the system detects such a trigger it preferably decides to ignore this rendering session. Some triggers may be difficult to detect automatically. It is thus preferred that the system takes the final decision (second decision) only based on several rendering sessions of the same song. A negative first indicator is then just a negative indicator and in itself will not result in a final rejection. Three successive negative indicators may, for example, be taken as a final negative indicator. The system may cope with such accidental negative indicator by, for example, on detecting a positive indicator that follows a previous negative indicator resetting the counter to one (i.e. one positive indicator). A negative first indicator followed by a second negative indicator would not result in resetting the counter. This enables the system to detect three successive negative first indicators and filters out an isolated negative first indicators. Persons skilled in the art will be able to design more advanced filtering schemes as the second criterion.

In a preferred embodiment, the memory 140 is arranged to store for the predetermined physiological state an associated playlist of songs for bringing the user closer to the predetermined physiological state. The playlist may take any suitable form, for example as a fully separate playlist or additional attributes or attribute values on an existing playlist. The user can then select songs for bringing him to the desired state simply by selecting the playlist (or equally, by selecting the desired state and filtering a larger playlist for that attribute). The second criterion can then be used for determining whether the song should be added to and/or maintained on the playlist. It will be appreciated that once a song has been ‘finally’ accepted on the playlist, that the system actually may keep on monitoring and in that way improve the selection process. A preferred way of doing this is to use a separate counter for the number of times Nₚₒₛ a first positive indicator has been given and the number of times N_{neg} a negative first indicator has been given. The total rating is then Nₚₒₛ/(Nₚₒₛ+N_{neg}).

In the embodiment where the outcome of the second criterion is more varied then just suitable/not suitable, preferably the processor is programmed to determine a relative rate of rendering of a song on the playlist in dependence on the associated suitability factor. A more suitable song is rendered more frequently than a less suitable song. Preferable a threshold is used for ensuring that a song with a suitability factor below the threshold is not rendered for this physiological state. The more songs have been tested, the stricter the selection criteria may become. For example, the threshold is raised. The embodiment of FIG. 3 shows that in step 310 it is checked whether or not the song should be rendered for the desired physiological state (e.g. whether or not the suitability factor is above/below the threshold). If the song should not be rendered a next song is selected in step 305. A representation of the suitability factor is stored in the memory in association with the song. It will be appreciated that the suitability factor can at least indicate: fully unsuitable, highly suitable and at least one intermediate level. Preferably more than these three stages are supported, for example ten stages. In the example given above, the suitability factor is Nₚₒₛ/(Nₚₒₛ+N_{neg}).

In an embodiment of the system, the memory includes a candidate playlist of songs being tested for acceptance on the playlist associated with the physiological state. The candidate play-list includes for songs on this candidate playlist associated information, such as the indicator indicating whether rendering of the song has a positive effect on relaxing the user. If the song is tested four times, the list may store for the song all four indicators. Alternatively, whenever a new indicator has been determined a new value to be stored is determined based on the stored indicator and the newly determined indicator. For example, one positive indicator may add 25% to the stored value. So, after four positive tests the outcome is 100% suitable. In this way, a four-valued ranking can be achieved in a simple way.

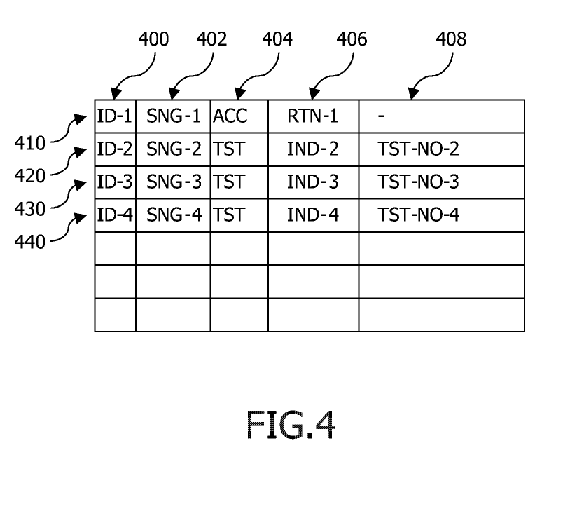

FIG. 4 shows an example of how information can be stored for playlists. In this example, for a specific physiological state the playlist combines songs that have already been accepted and songs that are being tested. Each row 410, 410, 430 and 440 represents a respective song on the playlist. Each column 400, 402, 404, 406, and 408 represents a field for storing data for the song. In field 400 an identifier for the song may be stored (e.g. a sequential number, a title, etc.). In field 402a reference is made to the actual song content (e.g. file indicator, such as file name and directory). Since in this example the list includes both accepted songs as well as songs being tested, field 404 is used for distinguishing between both types. Based on information in this field, the other field(s) may include information specific for each type of songs. For example, for an already accepted song field 406 may store the final rating (suitability factor). For a song being tested, field 406 may stored the accumulated (or last obtained) first indicator and field 408 may store the number of times the song has already been tested. The embodiment of FIG. 3 can use the information in field 404 to differentiate in step 320 whether the song is already accepted, followed by simply rendering the song in step 322, or by performing a testing, starting with step 324 as described above.

In an embodiment, the processor is programmed to use the brain-wave sensor to determine at least one of the following physiological states:

the user is falling asleep,

the user is asleep,

the user is awakening,

the user is alert;

and, in response to determining so, stop rendering of songs or start rendering of songs. For example, if the desired physiological state is ‘asleep’ and based on the measurement done during the rendering of the song it is determined that the user has by now fallen asleep, rendering of the songs may be stopped. If on the other hand, for the same desired state, it is detected that the user is awakening, rendering may be re-started.

The user interface 160 may be used for allowing the user to select the desired physiological state (if the particular system supports more than one state). Preferably, the system is also programmed to determine the desired physiological state automatically. For example, by receiving a trigger from an alarm set by the user, the system can automatically set the desired physiological state to alert. It can also do this if it detects that the user is moving (e.g. using movement sensors or GPS). It may also choose the desired physiological state in dependence on the time (e.g. late in the evening: asleep) and/or date (e.g. day of the week). Preferably, the system learns the desired physiological state from the user. For example, if the user during working days regularly around 18.00 o'clock activates the system and sets the desired state to ‘relaxed’ the system can perform this action automatically based on a signal from a clock.

It will be appreciated that the invention also extends to computer programs, particularly computer programs on or in a carrier, adapted for putting the invention into practice. The program may be in the form of source code, object code, a code intermediate source and object code such as partially compiled form, or in any other form suitable for use in the implementation of the method according to the invention. The carrier be any entity or device capable of carrying the program. For example, the carrier may include a storage medium, such as a ROM, for example a CD ROM or a semiconductor ROM, or a magnetic recording medium, for example a floppy disc or hard disk. Further the carrier may be a transmissible carrier such as an electrical or optical signal, which may be conveyed via electrical or optical cable or by radio or other means. When the program is embodied in such a signal, the carrier may be constituted by such cable or other device or means. Alternatively, the carrier may be an integrated circuit in which the program is embedded, the integrated circuit being adapted for performing, or for use in the performance of, the relevant method.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1-18. (canceled)

19. A system (100) for categorizing songs based on a physiological effect of the song on a user; the system including:
a storage medium (120) for storing a plurality of songs;
a memory (140) for storing an associated predetermined first selection criterion for at least one predetermined physiological state of a user, said memory also storing for each of at least one predetermined physiological state, an associated playlist of songs for bringing the user closer to a particular predetermined physiological state;
a rendering system (150) for rendering songs retrieved from the storage medium;
a sensor (130) for determining a biological parameter of the user representative of a physiological state of the user; and
a processor (110) configured for:
repeatedly causing the selected song to be rendered,
using the sensor to obtain at least one measurement during said rendering,
using the first predetermined criterion to determine, in dependence on the measurement, a respective indicator indicating whether rendering of the song has a positive effect on bringing the user closer to the predetermined physiological state; and
using a second predetermined criterion to determine, based on a plurality of the determined indicators for the song, a suitability of the song for bringing the user closer to the predetermined physiological state.

20. A system as claimed in claim 19, wherein the processor is programmed to use the sensor at least twice during the rendering of the song to obtain at least two time-sequential measurements of the biological parameter; the first criterion being dependent on a change of the at least two measurements.

21. A system as claimed in claim 19, wherein the sensor is a brain-wave sensor for determining a frequency of brainwaves of the user.

22. A system as claimed in claim 19, wherein the sensor is of a contact-free type.

23. A system as claimed in claim 20, wherein the predetermined physiological state is asleep and the first criterion includes whether a brainwave frequency has decreased during rendering of the song.

24. A system as claimed in claim 20, wherein the predetermined physiological state is alert and the first criterion includes whether a brainwave frequency has increased during rendering of the song.

25. A system as claimed in claim 19, wherein the first criterion is dependent on an actual physiological state being measured during the rendering

26. A system as claimed in claim 20, wherein the first criterion includes whether the measured physiological state is the predetermined physiological state and whether no substantial change has been detected between the at least two measurements during the rendering.

27. A system as claimed in claim 19, wherein the second criterion includes that a predetermined successive number of times during the rendering a same song all respective indicators have indicated that rendering of the song has a positive effect on bringing the user closer to the predetermined physiological state.

28. A system a claimed in claim 19, wherein the memory is arranged to store for the predetermined physiological state an associated playlist of songs for bringing the user closer to the predetermined physiological state.

29. A system as claimed in claim 28, wherein the processor is programmed to, based on the determined suitability, determining whether the song should be added to and/or maintained on the playlist.

30. A system as claimed in claim 28, wherein the memory includes for songs on the playlist an associated suitability indicator; and wherein the processor is programmed to determine a relative rate of rendering of a song on the playlist in dependence on the associated suitability factor.

31. A system as claimed in claim 28, wherein the memory includes a candidate playlist of songs being tested for acceptance on the playlist; the candidate play-list including for songs on the candidate playlist associated information including at least the indicator indicating whether rendering of the song has a positive effect on relaxing the user.

32. A system as claimed in claim 21, wherein the processor is programmed to use the brain-wave sensor to determine at least one of the following physiological states:
the user is falling asleep,
the user is asleep,
the user is awakening,
the user is alert;
and, in response to determining so, stop rendering of songs or start rendering of songs.

33. A system as claimed in claim 19, wherein the system includes a user interface for enabling a user to select the song for testing.

34. A system as claimed in claim 19, wherein the rendering system includes speakers for generating spatially localized sound, such as an earphone, headphone or sound-beamer.

35. A method of categorizing songs on a physiological effect of the song on a user; the method including:
repeatedly causing the selected song to be rendered (210) by a rendering system,
using a sensor to measure (210) at least once during the rendering of the song a biological parameter of the user representative of a physiological state of the user,
using a first predetermined criterion (220) associated with a predetermined physiological state of a user to determine, in dependence on the measurement, a respective indicator indicating whether rendering of the song has a positive effect on bringing the user closer to the predetermined physiological state; and
using a second predetermined criterion (240) to determine, based on a plurality of the determined indicators for the song, a suitability of the song for bringing the user closer to the predetermined physiological state.

36. A computer program product for causing a processor to perform the method of claim 35.
